(19) United States
(12) Patent Application Publication
(10) Pub. No.: US 2006/0257503 A1
(43) Pub. Date: Nov. 16, 2006

(54) Nutritional supplement and method for providing supplemental nutrition to an athlete

(75) Inventor: Gayle Eversole, Moscow, ID (US)

Correspondence Address: WELLS ST. JOHN P.S., 601 W. FIRST AVENUE, SUITE 1300, SPOKANE, WA, 99201, US

(73) Assignee: N22 Nutrition, LLC

(21) Appl. No.: 11/130,302

(22) Filed: May 16, 2005

(51) Int. Cl. A61K 31/44; A61K 31/455; A61K 31/198; A61K 31/19; A61K 33/06

(52) U.S. Cl. 424/682; 514/356; 514/574; 514/561

(57) ABSTRACT

A nutritional supplement is disclosed and which includes an ingestible mixture of Magnesium Chelate, Malic Acid, Dimethylglycine HCL and Niacinamide in amounts which, when ingested, reduces the occurrence of chronic exertional compartment syndrome in an athlete.

### TECHNICAL FIELD

The present invention relates to a nutritional supplement and to a method for providing supplemental nutrition to an athlete who will be engaging in prolonged vigorous athletic activity, and more specifically, to a composition which when orally ingested is substantially effective in reducing the occurrence of chronic exertional compartment syndrome in the forearm of the athlete.

### BACKGROUND OF THE INVENTION

The beneficial effects of providing various nutritional supplements to enhance the performance of athletes who are engaging in prolonged vigorous athletic activity has long been known. Examples of prior art references teaching various compositions which are useful in this regard include, but are not limited to, U.S. Pat. Nos. 4,322,407; 4,599,152; 4,725,427; 4,863,898; 5,270,297; 5,292,538; 6,245,378; 6,562,869; and Published U.S. Patent Application 2004/0204382. The teachings of these various prior art patents and the published patent application are incorporated by reference herein.

In the athletic event of motocross, motorcycle riders will drive specially designed motorcycles over various rough terrain and track configurations for prolonged periods of time. This event requires extreme amounts of upper body strength. Participants in this sport have long reported that they are often limited in their performance because of a periodic chronic disability often referred to as “arm pump.” “Arm pump” which is referred to in the medical literature as “chronic exertional compartment syndrome of the forearm” is a largely misunderstood malady which causes in some cases extreme pain, and prevents athletes from continuing to compete in events such as motocross once the disorder manifests itself. Typically, severe “arm pump” occurs as a result of the muscle exertion that the athlete exerts as he grips the handlebars of his motorcycle during the racing event. After a period of prolonged muscle exertion, the forearms of the athlete painfully enlarge and harden. Further, the athlete's hands may become substantially useless. As will be recognized, without use of the hands to hold onto the handlebars, and the fingers which are often needed for applying the brakes, and for shifting the motorcycle, the athlete cannot continue to compete in the motocross event and must often leave the track.

As should be understood, during vigorous exercise such as might be occasioned by the arm of an athlete in a motocross event, the muscles in the arm require a tremendous amount of oxygen rich blood. When engorged with blood, the forearms commonly increase in volume by up to 20%. The engorged muscle which is positioned inside an inelastic fascia then experiences increased pressure within the compartment of the forearm. As will be recognized, although gases and solids are compressible, fluids are not. The incompressible fluid (blood) within the inelastic fascia makes the forearm feel hard. If the compartment pressure within the forearm rises high enough, blood vessels can collapse within the forearm compartment which restricts or stops the flow of blood through those vessels. Veins, with their low pressure and thin walls, typically collapse earlier than high pressure, thick walled arteries. When venous blood flow reduces, arterial blood continues to enter the fascia compartment of the forearm, but is restricted from leaving. Typically, “arm pump” is relieved by stopping the activity and the forearm slowly returns to its normal state over a period of time.

While various surgical techniques have been proposed for minimizing the occurrence of “arm pump” or so-called “chronic exertional compartment syndrome of the forearm,” these have been less then satisfactory. Still further, and while various compositions as noted in the prior art references identified above have discussed various compositions which appear to be useful for increasing the endurance and health of an athlete, no prior art teachings are available which have suggested a composition which might be useful in any fashion to reduce the occurrences of this same malady.

A nutritional supplement and method associated with same which reduces the occurrence of chronic exertional compartment syndrome in an athlete is the subject matter of the present application.

### SUMMARY OF THE INVENTION

A first aspect of the present invention relates to a nutritional supplement which includes an ingestible mixture of Magnesium Chelate, Malic Acid, Dimethylglycine HCL and Niacinamide in an amount which, when ingested, reduces the occurrence of chronic exertional compartment syndrome in an athlete.

Another aspect of the present invention relates to a nutritional supplement which reduces the occurrence of chronic exertional compartment syndrome in the forearm of an athlete and which includes about 11.52% to about 17.28% by weight of Magnesium Chelate; about 52.29% to about 63.91% by weight of Malic Acid; about 2.74% to about 3.35%, by weight of Dimethylglycine HCL; and about 22.01% to about 26.89%, by weight of Niacinamide.

Another aspect of the present invention relates to a nutritional supplement which reduces the occurrence of chronic exertional compartment syndrome in an athlete during strenuous exercise and which includes about 9.64% to about 14.44%, by weight, of Magnesium Chelate; about 43.38% to about 53%, by weight of Malic Acid; about 0.64% to about 2.78%, by weight of Dimethylglycine HCL; about 18.11% to about 22.13%, by weight of Niacinamide; about 10.3% to about 12.58%, by weight of rice flour; and about 5.09% to about 6.22% by weight of Magnesium Stearate.

Another aspect of the present invention relates to a nutritional supplement which reduces the occurrence of chronic exertional compartment syndrome in the forearm of an athlete and which consists essentially of about 12.04%, by weight, of Magnesium Chelate 20%; about 48.19%, by weight, of Malic Acid; about 2.53%, by weight, of Dimethylglycine HCL; about 20.12%, by weight, of niacinamide; about 11.44%, by weight, of rice flour; and about 5.68%, by weight, of Magnesium Stearate.

Yet still another aspect of the present invention relates to a method for providing supplemental nutrition to an athlete who will be engaging in prolonged vigorous athletic activity and which includes, prior to engaging in the prolonged vigorous athletic activity, a step of ingesting a biologically effective amount of a Magnesium Chelate; Malic acid; Niacinamide; and Dimethylglycine HCL and which is substantially effective in reducing the occurrence of chronic exertional compartment syndrome in the forearm of the athlete.

These and other aspects of the present invention will be discussed in greater detail hereinafter.

### DETAILED DESCRIPTION OF THE PREFERRED EMBODIMENTS

This disclosure of the invention is submitted in furtherance of the constitutional purposes of the U.S. Patent Laws “to promote the progress of science and useful arts” (Article 1, Section 8).

The present invention relates to a nutritional supplement which comprises an ingestible mixture of Magnesium Chelate, Malic Acid, Dimethylglycine HCL and Niacinamide in respective amounts which, when ingested, reduces the occurrence of chronic exertional compartment syndrome in an athlete as earlier described. In the present invention, it should be understood that the nutritional supplement is typically provided in an ingestible capsule which is swallowed by the athlete in advance of an athletic event. Typically, a suitable amount is ingested at least about 30 minutes before an athletic event in order to curtail the occurrence of chronic exertional compartment syndrome. Still further, the present invention may be formulated in a fashion whereby it may be combined with a source of water and then drunk by the athlete prior to the athletic event. The capsule encloses biologically effective amounts of the respective materials such that no more than 9 capsules should be ingested in any 24 hour period. Typically, 3 capsules are ingested before the athletic event.

The Magnesium Chelate 20% which forms one of the active ingredients of the present invention is typically provided in an amount of at least about 9% by weight of the nutritional supplement, but more often, and when provided in an ingestible capsule form, is in a range of about 11.52% to about 17.28% by weight. Those skilled in the art understand that Magnesium Chelates have been identified as having numerous essential roles in energy and energy metabolism. Examples of U.S. Patents discussing the role of Magnesium and Magnesium Chelates of various types are seen in U.S. Pat. Nos. 5,270,297 and 5,292,538. Still further, U.S. Published Patent Application 2004/0204382 discusses the role of Magnesium and Magnesium Amino Acid Chelate in various biological functions including the metabolic cycle of ATP Production and Hydrolysis. Additionally, Magnesium is needed by the human body for the maintenance of bone, the use of proteins, and the production of fatty acids.

The nutritional supplement of the present invention further has a biologically effective amount of Malic Acid which is greater than about 43%, by weight, of the nutritional supplement. When formulated in the form of an ingestible capsule, the Malic Acid typically comprises about 52.29% to 63.91%, by weight. Those skilled in the art will recognize that Malic Acid is present in substantially all living cells, and is typically essential for manufacturing ATP which is a human body's energy source. Still further, Malic Acid has been implicated in proper muscle function and further has been identified as part of the Krebs Cycle which is employed for efficient energy production. Malic Acid is a naturally occurring fruit acid which is derived typically from fruit such as apples. In the Krebs cycle, Malic Acid is typically considered to be a catalyst and which is employed with fats and glucose where they are converted during that cycle into energy. Still further, Malic Acid has been implicated as being a potent aluminum detoxifier and especially effective at decreasing aluminum toxicity in various organs and tissues including the brain.

The present nutritional supplement further includes an effective amount of Dimethylglycine HCL (DMG) which is greater than about 0.6% of the nutritional supplement. When formulated in the form of an ingestible capsule, the Dimethylglycine HCL is typically in a range of about 2.74% to about 3.35% by weight. Dimethylglycine has been referred to in the literature, heretofore, as Pangamic Acid or Vitamin B15. This compound was discovered by Dr. E. T. Krebs, Jr. while exploring the chemical properties of apricot kernels in 1952. Krebs viewed Vitamin B15 as a compound that would provide “instant oxygen”. Krebs considered the newly discovered substance as being capable of increasing the oxygen efficiency of the entire body. Further, he conjectured that B15 aided in the detoxification of waste products. Dimethylglycine, in its various forms, has been taught in the prior art as being useful in the treatment of various maladies including various forms of cancers; inflammation of connective tissue occurring as a result of such diseases as arthritis; boosting a mammals immune response; and for the treatment of seizure disorders such as epilepsy. See U.S. Pat. Nos. 4,994,492; 4,385,068; 5,026,728 and 4,595,697. Much anecdotal evidence produced from such sources as Russian athletes and cosmonauts who utilized Dimethylglycine HCL to improve athletic performance or on extended space missions has been published. However, claims that Dimethylglycine HCL (DMG) can boost energy and athletic performances have been refuted by some human and animal studies. Other investigators have indicated that DMG may be responsible for decreasing lactate in the blood, and increasing creatine phosphate in the body. The substance may also be considered a liprotropic (fat dissolving) factor.

The present nutritional supplement further includes an effective amount of Niacinamide (Vitamin B3) which is greater than about 18% by weight of the nutritional supplement. When formulated as an ingestible capsule, the amount of Niacinamide (B3) is about 18.11% to about 22.13%, by weight. The various B vitamins have been implicated in various biological functions in the body and are variously discussed in the prior art references identified, above, all of which have been incorporated by reference herein. Niacinamide has been demonstrated to play roles in the energy producing Glycolysis cycle. Further, Niacinamide can increase energy in the human body through improving the utilization of food. Additionally, Niacinamide has been implicated, in the form of Nicotinic Acid, in regulating blood sugar levels as part of the glucose tolerance factor. Niacinamide can be assisted by the presence of Chromium, Magnesium and/or Manganese. Moreover, this same vitamin appears to facilitate capillary circulation and thereby enhances blood flow to the muscle tissue. Enhanced blood flow, of course, increases the delivery of oxygen and other nutrients to the tissue. The use of various other B vitamins in various energy producing compositions are taught in Published U.S. Patent Application 2004/0204382, the teachings of which are incorporated by reference herein.

As discussed above, when formulated into an ingestible capsule, the Niacinamide may constitute about 22.01% to about 26.89% by weight.

As referenced, above, the nutritional supplement may be manufactured in the form of an ingestible capsule which includes, among the other ingredients noted above, filler materials of various types and amounts, and which are well known in the prior art and which are typically considered inert. For example, in the formulation of the nutritional supplement of the present invention, the nutritional supplement may include about 10.3% to about 12.58%, by weight, of a rice flour; and about 5.09% to about 6.22%, by weight, of Magnesium Stearate. These materials are frequently used in capsules of various types. For example, rice flour is often used to reduce the adverse effects related to allergies. Magnesium Stearate and rice flour are typically utilized in amounts so as to permit the other ingredients to flow more easily during the machine encapsulating process. The ingestible capsules noted above, typically enclose about 0.1 mg. of the Magnesium Chelate 20%; about 0.4 mg, of the Malic Acid; about 0.021 mg. of the Dimethylglycine HCL; about 0.167 mg. of the Niacinamide; about 0.095 mg. of the rice flour; and about 0.047 mg. of the Magnesium Stearate. In the amounts, as noted above, the nutritional supplement which reduces the occurrence of chronic exertional compartment syndrome in the forearm of an athlete consists essentially of about 12.04%, by weight, of Magnesium Chelate 20%; about 48.19%, by weight, of Malic Acid; about 2.53%, by weight, of Dimethylglycine HCL; about 20.12%, by weight, of niacinamide; about 11.44%, by weight, of rice flour; and about 5.68%, by weight, of Magnesium Stearate.

The present invention also relates to a method for providing supplemental nutrition to an athlete who will be engaging in prolonged vigorous athletic activity which includes, prior to engaging in the prolonged vigorous athletic activity, ingesting a biologically effective amount of a Magnesium Chelate; Malic acid; Niacinamide; and Dimethylglycine HCL and which is substantially effective in reducing the occurrence of chronic exertional compartment syndrome in the forearm of the athlete. In the methodology as described above, the biologically effective amount of the several substances noted in the sentence above is ingested at least about 30 minutes before engaging in the prolonged vigorous athletic activity. Still further, in the present methodology, after engaging in the vigorous activity, it has been found useful to ingest a biologically effective amount of the Magnesium Chelate; Malic acid; Niacinamide; and Dimethylglycine HCL and which is substantially effective in reducing the occurrence of chronic exertional compartment syndrome of the forearm of the athlete following the athletic activity. The method, as described above, further includes the steps of providing about 9.64% to about 14.44%, by weight, of the Magnesium Chelate; providing about 43.38% to about 53%, by weight, of the Malic acid; providing about 0.64% to about 2.78%, by weight, of the Dimethylglycine HCL; providing about 18.11% to about 22.13%, by weight, of the Niacinamide; providing about 10.3% to about 12.58%, by weight, of a filler material which comprises a rice flour; providing about 5.09% to about 6.22%, by weight, of a filler material which comprises Magnesium Stearate; mixing the Magnesium Chelate; Malic Acid; Dimethylglycine HCL; Niacinamide; rice flour; and Magnesium Stearate to provide a substantially homogeneous mixture; and enclosing the substantially homogeneous mixture in an ingestible capsule.

The present nutritional supplement which has been used by numerous motocross riders has repeatedly been shown to reduce the effects of calcium buildup and phosphate accumulation that leads to muscle fatigue and decreased performance especially as it relates to the onset of chronic exertional compartment syndrome in an athlete. The present formulation is effective and reduces the onset of chronic exertional compartment syndrome in athletes and particularly the manifestation of the malady in the forearms of athletes, especially those who participate in activities such as motocross competition.

In compliance with the statute, the invention has been described in language more or less specific as to structural and methodical features. It is to be understood, however, that the invention is not limited to the specific features shown and described, since the means herein disclosed comprise preferred forms of putting the invention into effect. The invention is, therefore, claimed in any of its forms or modifications within the proper scope of the appended claims appropriately interpreted in accordance with the doctrine of equivalents.

I claim:

1. A nutritional supplement, comprising:
an ingestible mixture of Magnesium Chelate, Malic Acid, Dimethylglycine HCL and Niacinamide in an amount which, when ingested, reduces the occurrence of chronic exertional compartment syndrome in an athlete.

2. A nutritional supplement as claimed in claim 1, and wherein the nutritional supplement is provided in an ingestible capsule which is swallowed by the athlete.

3. A nutritional supplement as claimed in claim 1, and wherein the nutritional supplement is combined with water and then drunk by the athlete.

4. A nutritional supplement as claimed in claim 1 and wherein an effective amount of the Magnesium Chelate is greater than about 9%, by weight, of the nutritional supplement.

5. A nutritional supplement as claimed in claim 1, and wherein an effective amount of the Malic Acid is greater than about 43%, by weight, of the nutritional supplement.

6. A nutritional supplement as claimed in claim 1, and wherein an effective amount of the Dimethylglycine HCL is greater than about 0.6%, by weight, of the nutritional supplement.

7. A nutritional supplement as claimed in claim 1, and wherein an effective amount of the Niacinamide is greater than about 18%, by weight, of the nutritional supplement.

8. A nutritional supplement as claimed in claim 1, and wherein the chronic exertional compartment syndrome is experienced in the forearm of the athlete.

9. A nutritional supplement which reduces the occurrence of chronic exertional compartment syndrome in the forearm of an athlete, comprising:
about 11.52% to about 17.28% by weight of Magnesium Chelate;
about 52.29% to about 63.91% by weight of Malic Acid;
about 2.74% to about 3.35%, by weight of Dimethylglycine HCL; and
about 22.01% to about 26.89%, by weight of Niacinamide.

10. A nutritional supplement which reduces the occurrence of chronic exertional compartment syndrome in an athlete during strenuous exercise, comprising:
about 9.64% to about 14.44%, by weight, of Magnesium Chelate;
about 43.38% to about 53%, by weight of Malic Acid;
about 0.64% to about 2.78%, by weight of Dimethylglycine HCL;
about 18.11% to about 22.13%, by weight of Niacinamide;
about 10.3% to about 12.58%, by weight of rice flour; and
about 5.09% to about 6.22% by weight of Magnesium Stearate.

11. A nutritional supplement as claimed in claim 10, and wherein the nutritional supplement is enclosed within an ingestible capsule.

12. A nutritional supplement as claimed in claim 11, and wherein no more than 3 capsules are ingested at least 30 minutes before, and immediately after strenuous exercise.

13. A nutritional supplement as claimed in claim 12, and wherein no more than 9 capsules are ingested in a 24 hour period.

14. A nutritional supplement as claimed in claim 11, and wherein the chronic exertional compartment syndrome occurs in the forearm of the athlete.

15. A nutritional supplement which reduces the occurrence of chronic exertional compartment syndrome in the forearm of an athlete, consisting essentially of:
about 12.04%, by weight, of Magnesium Chelate 20%;
about 48.19%, by weight, of Malic Acid;
about 2.53%, by weight, of Dimethylglycine HCL;
about 20.12%, by weight, of Niacinamide;
about 11.44%, by weight, of rice flour; and
about 5.68%, by weight, of Magnesium Stearate.

16. A nutritional supplement as claimed in claim 15, and wherein the nutritional supplement is enclosed within an ingestible capsule, and wherein each capsule encloses about 0.1 mg. of the Magnesium Chelate 20%; about 0.4 mg, of the Malic Acid; about 0.021 mg. of the Dimethylglycine HCL; about 0.167 mg. of the Niacinamide; about 0.095 mg. of the rice flour; and about 0.047 mg. of the Magnesium Stearate.

17. A method for providing supplemental nutrition to an athlete who will be engaging in prolonged vigorous athletic activity, comprising:
prior to engaging in the prolonged vigorous athletic activity, ingesting a biologically effective amount of a Magnesium Chelate; Malic acid; Niacinamide; and
Dimethylglycine HCL and which is substantially effective in reducing the occurrence of chronic exertional compartment syndrome in the forearm of the athlete.

18. A method as claimed in claim 17, and wherein the biologically effective amount of Magnesium Chelate; Malic acid; Niacinamide; and Dimethylglycine HCL is ingested at least about 30 minutes before engaging in the prolonged vigorous athletic activity.

19. A method as claimed in claim 17, and further comprising:
after engaging in the vigorous athletic activity, ingesting a biologically effective amount of Magnesium Chelate; Malic acid; Niacinamide; and Dimethylglycine HCL which is substantially effective in reducing the occurrence of chronic exertional compartment syndrome of the forearm of the athlete.

20. A method as claimed in claim 17, and wherein the step of ingesting a biologically effective amount of a Magnesium Chelate; Malic acid; Niacinamide; and
Dimethylglycine HCL further comprises:
providing about 11.52% to about 17.28%, by weight, of Magnesium Chelate 20%;
providing about 52.29% to about 63.91%, by weight, of Malic acid;
providing about 2.74% to about 3.35%, by weight, of Dimethylglycine HCL; and
providing about 22.01% to about 26.89%, by weight, on Niacinamide.

21. A method as claimed in claim 17, and further comprising:
providing about 9.64% to about 14.44%, by weight, of the Magnesium Chelate;
providing about 43.38% to about 53%, by weight, of the Malic acid;
providing about 0.64% to about 2.78%, by weight, of the Dimethylglycine HCL;
providing about 18.11% to about 22.13%, by weight, of the Niacinamide;
providing about 10.3% to about 12.58%, by weight, of a filler material which comprises a rice flour;
providing about 5.09% to about 6.22%, by weight, of a filler material which comprises Magnesium Stearate;
mixing the Magnesium Chelate; Malic Acid; Dimethylglycine HCL; Niacinamide;
rice flour; and Magnesium Stearate to provide a substantially homogeneous mixture; and
enclosing the substantially homogeneous mixture in an ingestible capsule.
